(19) United States
(12) Patent Application Publication
(10) Pub. No.: US 2022/0232821 A1
(43) Pub. Date: Jul. 28, 2022

(54) RED BLOOD CELL STORAGE SOLUTIONS, ADDITIVES, AND METHODS FOR IMPROVING THE STORAGE OF RED BLOOD CELLS USING INORGANIC PYROPHOSPHATES

(71) Applicant: University of Cincinnati, Cincinnati, OH (US)

(72) Inventors: Timothy A. Pritts, Cincinnati, OH (US); Kasiemobi Pulliam, Cincinnati, OH (US); Bernardin Joseph, Cincinnati, OH (US); Charles Caldwell, Cincinnati, OH (US); Alex B. Lentsch, Cincinnati, OH (US); Amy T. Makley, Cincinnati, OH (US); Michael D. Goodman, Cincinnati, OH (US)

(21) Appl. No.: 17/617,191

(22) PCT Filed: Jun. 5, 2020
(86) PCT No.: PCT/US2020/036233

§ 371 Date: Dec. 7, 2021

Related U.S. Application Data
(60) Provisional application No. 62/858,576, filed on Jun. 7, 2019.
(60) Provisional application No. 62/859,257, filed on Jun. 10, 2019.

(51) Int. Cl. A01N 1/02; C12N 5/078

(52) U.S. Cl. CPC A01N 1/0226; C12N 5/0641

(57) ABSTRACT

Solutions are provided herein for the collection of whole blood and/or storage of packed red blood cells, the solutions including one or more inorganic pyrophosphates (PPi). Also provided are methods of storing whole blood or packed red blood cells and methods of mitigating a complication associated with a transfusion or infusion of whole blood or red blood cells, the methods including storing the whole blood or red blood cells in a solution including one or more inorganic pyrophosphates.

### CROSS-REFERENCE TO RELATED APPLICATIONS

This application claims priority to U.S. Provisional Application No. 62/858,576 filed Jun. 7, 2019 and U.S. Provisional Application No. 62/859,257 filed Jun. 10, 2019, the entire contents of which are incorporated by reference herein.

### STATEMENT REGARDING FEDERALLY SPONSORED RESEARCH OR DEVELOPMENT

This invention was made with government support under R01GM107625 awarded by the National Institutes of Health. The government has certain rights in the invention.

### FIELD

This disclosure relates to the field of blood product storage solutions. Specifically, this disclosure relates to blood collection solutions and packed red blood cell storage solutions and methods for improved collection and storage of red blood cells.

### BACKGROUND

Hemorrhagic shock is the most common cause of preventable death in traumatically injured patients and transfusion of human blood products, including stored/packed red blood cell units (pRBCs), is the ideal treatment for hemorrhagic shock in the acute setting. Unfortunately, this use of blood products may result in later harm to the patient. Liberal transfusion strategies with use of pRBCs to treat anemia have been associated with poor clinical outcomes and increased mortality in critically ill patients, an effect that is thought to be related, at least in part, to age of pRBCs. Compared to fresh units, transfusion of aged pRBCs has been associated with increased rates of pneumonia, sepsis, multi-organ failure, and mortality.

Standard blood banking inventory management relies on a “first in, first out” system, whereby the oldest viable pRBC units are often used first. Thus, the average age of transfused pRBCs ranges from 20-30 or more days. As the life span of erythrocytes in the circulating system is 120 days, patients receiving pRBC transfusion at the end of the current FDA shelf life of 42 days of storage may be exposed to erythrocytes that range in age from 42 to 162 days in chronological age.

As pRBCs age, they develop changes in biochemical and molecular parameters known as the red blood cell or erythrocyte “storage lesion.” Erythrocyte structural proteins, lipids, and carbohydrates undergo oxidative injury which leads to cross-linking of erythrocyte membrane phospholipids and proteins. Alterations in membrane structural components, including the spectrin-actin-protein 4.1 complex and band 3, result in loss of membrane domain as well as the classic erythrocyte biconcave disc shape, with subsequent appearance of echinocytes and spherocytes as well as a loss of normal deformability. Phosphatidylserine, normally on the intracellular side of the plasma membrane, becomes externalized. These membrane changes and increased fragility contribute to increased acidosis and hemolysis observed during the storage of pRBCs, as well as decreased erythrocyte survival following transfusion.

During storage, pRBCs also generate microvesicles (MVs, also referred to as microparticles, or MPs). Aged pRBCs cause increased lung microvascular permeability and neutrophil migration compared to fresh pRBCs, which may be due to MV accumulation in aged blood. In mouse and rat models, washing aged pRBCs to remove MVs prior to transfusion has been shown to diminish lung injury. Transfusion of aged pRBCs has also been clinically associated with increased incidence of deep vein thrombosis. Elevated levels of erythrocyte-derived MVs in patients have been associated with increased thrombin formation and complement activation. MVs from aged pRBCs have been shown in vitro to induce thrombin generation, potentially due to increased phosphatidylserine expression or interactions with factor XII. MVs isolated from aged pRBCs have also been shown to promote development of pulmonary microthrombi in mouse models of transfusion.

Under normal flow conditions, erythrocytes, usually 6-9 μm in diameter, are able to flex their discoid shape in order to squeeze through capillary vessels that are only 3-6 μm wide. The decreased deformability of aged erythrocytes leads to reduced capillary flow, decreased oxygen delivery to tissues, and impaired survival of transfused erythrocytes. Aged erythrocytes have also been shown to have increased adhesion to endothelial cells, likely due to the increased phosphatidylserine on the external erythrocyte membrane.

Increased free-hemoglobin in stored pRBCs further exacerbates this microangiopathy by scavenging nitric oxide (NO), which is generated by endothelial cells and helps control blood flow by inducing relaxation of vascular smooth muscle. As a result, transfusion of pRBCs with age-related hemolysis impedes endothelial-dependent vasodilation and end organ perfusion, which may have a significant a clinical impact. Decreased cerebral perfusion may be a reason that cardiac surgery patients receiving older pRBCs are at an increased risk for post-operative delirium. In rat models, transfusion of aged pRBCs has negative effects on liver perfusion and necrosis and leads to acute hypertension, vascular injury, and kidney dysfunction. Similarly, it has been demonstrated that an increase in pulmonary artery pressure following transfusion of old blood was mitigated with inhaled NO. Given that one pRBC contains 220-250 mg of iron, transfusion recipients must rapidly clear greater than 50 mg of iron per pRBC transfused. Following transfusion, iron deposition is visibly evident in the liver, spleen, and kidney. Clinical studies have correlated age of pRBCs transfused, as well as number of units transfused, with infectious complications. Preventing hemolysis in stored pRBCs would offer significant benefit to transfusion recipients.

Use of older units of pRBCs in the setting of trauma, hemorrhage, and resuscitation is associated with harm to the recipient, including increased risk of organ failure, renal failure, infection, venous thrombosis, and death. Although administration of fresh pRBC units would decrease potential harm from stored pRBCs, loss of inventory and the logistics of long distance transportation in rural and military settings render this solution difficult to implement.

A need exists for improved solutions and methods for collecting whole blood and storing pRBCs in order to preserve cell quality and reduce the incidence of harm after transfusion.

### SUMMARY

Accordingly, provided herein are solutions for the collection of whole blood and/or storage of packed red blood cells comprising one or more inorganic pyrophosphates (PPᵢ). Advantageously, the present investigators have found that anticoagulant collection and storage solutions comprising one or more derivatives of PPᵢ provide for decreased microvesicle accumulation, increased red blood cell quality, and decreased risk of adverse effects associated with whole blood or pRBC transfusion.

In one embodiment, a packed red blood cell (pRBC) storage solution is provided, the solution comprising one or more inorganic pyrophosphates.

In another embodiment, an anticoagulant blood collection solution is provided, the solution comprising one or more inorganic pyrophosphates.

In another embodiment, a method of preserving red blood cells is provided, the method comprising storing whole blood or packed red blood cells (pRBCs) in a solution comprising one or more inorganic pyrophosphates.

In another embodiment, a method for mitigating a complication associated with a transfusion or infusion of red blood cells is provided, the method comprising storing the red blood cells in a solution comprising one or more inorganic pyrophosphates.

These and additional aspects and features of the instant disclosure will be clarified by reference to the figures and detailed description set forth herein.

### BRIEF DESCRIPTION OF THE DRAWINGS

FIG. 1. Aspects of the red blood cell storage lesion in human packed red blood cell units (pRBCs) stored in AS-3 or PPi-3 for 42 days. (A) Microvesicles, (B) Cell-free hemoglobin in the supernatant of stored units, (C), Band-3 expression, and (D) Phosphatidylserine externalization. N≥5. *p<0.05 by t-test as indicated.

FIG. 2. Aspects of the red blood cell storage lesion in human packed red blood cell units (pRBCs) stored in AS-3 or PPi-3 for 42 days. (A) Osmotic fragility as determined by hemolysis in decreasing concentrations of salt solution. (B) EC 50 for the two storage solutions. (C) Red blood cell count at the end of the storage period. (D) Supernatant hemoglobin in the stored units. N≥5. *p<0.05 by t-test as indicated.

FIG. 3. Aspects of the red blood cell storage lesion in human packed red blood cell units (pRBCs) stored in AS-3 or PPi-3 for 42 days. (A) ATP content in stored erythrocytes. (B) glucose consumption during storage. N≥5. *p<0.05 by t-test as indicated.

FIG. 4. Aspects of the red blood cell storage lesion in murine packed red blood cell units (pRBCs) stored in AS-3 or PPi-3 for 14 days. (A) Microvesicles, (B) Cell-free hemoglobin in the supernatant of stored units, (C), Band-3 expression, and (D) Phosphatidylserine externalization. N≥5. *p<0.05 by t-test as indicated.

FIG. 5. Aspects of the red blood cell storage lesion in murine packed red blood cell units (pRBCs) stored in AS-3 or PPi-3 for 14 days. (A) Osmotic fragility as determined by hemolysis in decreasing concentrations of salt solution. (B) EC 50 for the two storage solutions. (C) micrographs of erythrocytes from each storage solution. (C) Flow cytometry examining cellular complexity. N≥5. *p<0.05 by t-test as indicated.

FIG. 6. Aspects of the red blood cell storage lesion in murine packed red blood cell units (pRBCs) stored in AS-3 or PPi-3 for 14 days. (A) Red blood cell count. (B) Erythrocyte hemoglobin content. (C) Glucose consumption during storage. N≥5. *p<0.05 by t-test as indicated.

FIG. 7. Hemorrhage and resuscitation of mice with pRBC units stored in either AS-3 or PPi-3 for 14 days. (A) Mean arterial pressure (MAP) during hemorrhage and resuscitation. (B) Serum levels of the chemokine MIP-1α. (C) Serum measurements of free hemoglobin after resuscitation. N≥5. *p<0.05 by t-test as indicated.

FIG. 8. Inflammatory mediator levels in cell culture media of cultures lung endothelial cells following treatment with media (negative control), tumor necrosis factor alpha (TNF-α; positive control), or murine pRBCs stored in either AS-3 or PPi-3 for 24 hours. (A) Keratinocyte chemokine (KC). (B) Monocyte chemoattractant protein-1 (MCP-1/CCL2). (C) Cell-free hemoglobin. N≥5. *p<0.05 by t-test as indicated.

FIG. 9. Microparticle concentration in murine pRBCs stored in AS-3, PPi-3_{H}, or PPi-3_{L} for 14 days.

FIG. 10. Band-3 expression in murine pRBCs stored in AS-3, PPi-3_{H}, or PPi-3_{L} for 14 days.

FIG. 11. Annexin V expression in murine pRBCs stored in AS-3, PPi-3_{H}, or PPi-3_{L} for 14 days.

FIG. 12. Advanced oxidative protein product concentration (AOPP) in murine pRBCs stored in AS-3, PPi-3_{H}, or PPi-3_{L} for 14 days.

FIG. 13. Free hemoglobin in supernatant of murine pRBCs stored in AS-3, PPi-3_{H}, or PPi-3_{L} for 14 days.

FIG. 14. Erythrocyte hemoglobin content in murine pRBCs stored in AS-3, PPi-3_{H}, or PPi-3_{L} for 14 days.

FIG. 15. Base deficit in murine pRBCs stored in AS-3, PPi-3_{H}, or PPi-3_{L} for 14 days.

FIG. 16. Murine RBC morphology after storage in AS-3, PPi-3_{H}, or PPi-3_{L} for 14 days. Left panel shows micrographs of erythrocytes stored in AS-3 (top) or PPi-3_{H}(bottom). Right panel shows flow cytometry results of forward scatter (top) and side scatter (bottom) for each of PPi-3_{L}, PPi-3_{H}, and AS-3 at day 14.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, osmolarity, osmolality, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Provided herein are solutions for the anticoagulant collection of whole blood and/or storage of packed red blood cells comprising one or more inorganic pyrophosphates (PPi) or derivatives thereof. Advantageously, the present investigators have found that anticoagulant collection and storage solutions comprising one or more inorganic pyrophosphates provide for decreased microvesicle accumulation, increased red blood cell quality, and decreased risk of adverse effects associated with transfusion of blood, including transfusion or infusion of aged RBCs.

### Inorganic Pyrophosphates

Pyrophosphates, alternatively referred to as diphosphates, refer to phosphorus oxyanions that contain two phosphorus atoms in a P—O—P linkage:

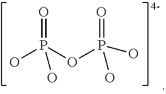

The anticoagulant collection and storage solutions disclosed herein comprise inorganic pyrophosphate (PPi) compounds, illustratively including salts, hydrates, esters, and other derivatives of inorganic pyrophosphates. Various suitable inorganic pyrophosphate salts are known in the art, including but not limited to: monosodium diphosphate, disodium diphosphate, trisodium diphosphate, tetrasodium pyrophosphate (also referred to as sodium pyrophosphate, tetrasodium phosphate, or TSPP), and the like. In embodiments, the inorganic pyrophosphate comprises anhydrous or hydrate forms of the inorganic pyrophosphate. Hydrate forms include, but are not limited to, disodium diphosphate hexahydrate, trisodium diphosphate nonahydrate, tetrasodium pyrophosphate decahydrate, and the like. In specific embodiments, the inorganic pyrophosphate is tetrasodium pyrophosphate or tetrasodium pyrophosphate decahydrate. In very specific embodiments, the inorganic pyrophosphate is tetrasodium pyrophosphate. In embodiments, the inorganic pyrophosphate comprises a mixture of any one or more inorganic pyrophosphates, salts, hydrates, esters, or derivatives thereof.

Inorganic pyrophosphates are present in the disclosed solutions at concentrations ranging from about 1 mM to about 200 mM, from about 1 mM to about 150 mM, from about 1 mM to about 100 mM, from about 1 mM to about 90 mM, from about 1 mM to about 80 mM, from about 1 mM to about 70 mM, from about 1 mM to about 60 mM, from about 1 mM to about 50 mM, from about 1 mM to about 40 mM, from about 1 mM to about 30 mM, from about 1 mM to about 25 mM, from 1 mM to about 20 mM, 2 mM to about 200 mM, from about 2 mM to about 150 mM, from about 2 mM to about 100 mM, from about 2 mM to about 90 mM, from about 2 mM to about 80 mM, from about 2 mM to about 70 mM, from about 2 mM to about 60 mM, from about 2 mM to about 50 mM, from about 2 mM to about 40 mM, from about 2 mM to about 30 mM, from about 2 mM to about 25 mM, from 2 mM to about 20 mM, from about from about 5 mM to about 200 mM, from about 5 mM to about 150 mM, from about 5 mM to about 100 mM, from about 5 mM to about 90 mM, from about 5 mM to about 80 mM, from about 5 mM to about 70 mM, from about 5 mM to about 60 mM, from about 5 mM to about 50 mM, from about 5 mM to about 40 mM, 5 mM to about 30 mM, from about 5 mM to about 25 mM, from about 10 mM to about 200 mM, from about 10 mM to about 150 mM, from about 10 mM to about 100 mM, from about 10 mM to about 90 mM, from about 10 mM to about 80 mM, from about 10 mM to about 70 mM, from about 10 mM to about 60 mM, from about 10 mM to about 50 mM, from about 10 mM to about 40 mM, from about 10 mM to about mM, from about 10 mM to about 20 mM, from about 20 mM to about 60 mM, from about 20 mM to about 50 mM, from about 20 mM to about 40 mM, from about 20 mM to about 30 mM, from about 30 mM to about 60 mM, from about 30 mM to about 50 mM, from about 30 mM to about 40 mM, from about 40 mM to about 60 mM, from about 40 mM to about 50 mM, or from about 50 mM to about 60 mM.

In embodiments, the solution is an anticoagulant blood collection solution and the concentration of inorganic pyrophosphate ranges from about 2 mM to about 100 mM, from about 5 mM to about 60 mM, from about 10 mM to about 60 mM, from about 20 mM to about 60 mM, from about 30 mM to about 60 mM, from about 30 mM to about 50 mM, or from about 30 mM to about 40 mM. In a specific embodiment, the anticoagulant blood collection solution has a concentration of inorganic pyrophosphate of from about 35 mM to about 45 mM or from about 35 mM to about 40 mM. In a specific embodiment, the anticoagulant blood collection solution has a concentration of inorganic pyrophosphate of about 37 mM. In a very specific embodiment, the anticoagulant blood collection solution has a concentration of tetrasodium pyrophosphate of about 37 mM.

In other embodiments, the solution is a pRBC storage solution and the concentration of inorganic pyrophosphate ranges from about 2 mM to about 100 mM, from about 5 mM to about 60 mM, from about 10 mM to about 60 mM, 5 mM to about 30 mM, from about 5 mM to about 25 mM, from about 10 mM to about 30 mM, or from about 10 mM to about 25 mM. In a specific embodiment, the pRBC storage solution has a concentration of inorganic pyrophosphate ranging from about 20 mM to about 25 mM. In a specific embodiment, the pRBC storage solution has a concentration of inorganic pyrophosphate of about 24 mM. In a very specific embodiment, the pRBC storage solution has a concentration of tetrasodium pyrophosphate of about 24 mM

### Additional Solution Components

In embodiments, the whole blood anticoagulant collection solutions and pRBC storage solutions disclosed herein may further comprise additional components, such as one or more of saline, electrolytes, nucleosides, sugars, or other additives.

In embodiments, the presently disclosed solutions may further comprise one or more electrolytes, illustratively selected from the group consisting of sodium chloride, monosodium phosphate, sodium citrate, sodium phosphate, sodium bicarbonate, sodium carbonate, calcium chloride, and combinations thereof.

In embodiments, the presently disclosed solutions may comprise a concentration of monosodium phosphate (NaH₂PO₄) of from about 0 mM to about 150 mM, from about 1 mM to about 150 mM, from about 1 mM to about 125 mM, from about 1 mM to about 100 mM, from about 1 mM to about 80 mM, from about 1 mM to about 70 mM, from about 1 mM to about 60 mM, from about 1 mM to about 50 mM, from about 1 mM to about 40 mM, from about 1 mM to about 30 mM, from about 1 mM to about 25 mM, from about 1 mM to about 20 mM, from about 1 mM to about 15 mM, from about 1 mM to about 10 mM, from about 2 mM to about 10 mM, about 1 mM, about 2 mM, about 3 mM, about 4 mM, about 5 mM, about 6 mM, about 7 mM, about 8 mM, about 9 mM, or about 10 mM.

In a specific embodiment, the solution is an anticoagulant collection solution and the concentration of monosodium phosphate (NaH₂PO₄) ranges from about 0 mM to about 150 mM, from about 10 mM to about 150 mM, from about 10 mM to about 120 mM, from about 20 mM to about 120 mM, from about 30 mM to about 120 mM, from about 40 mM to about 120 mM, from about 50 mM to about 120 mM, from about 60 mM to about 120 mM, from about 70 mM to about 120 mM, from about 80 mM to about 120 mM, from about 90 mM to about 120 mM, or from about 100 mM to about 120 mM. In a specific embodiment, the concentration of monosodium phosphate ranges from about 100 mM to about 115 mM. In a very specific embodiment, the concentration of monosodium phosphate in the anticoagulant collection solution is about 112 mM.

In another specific embodiment, the solution is a pRBC storage solution and the concentration of monosodium phosphate ranges from about 0 mM to about 20 mM, from about 1 mM to about 20 mM, from about 1 mM to about 15 mM, from about 1 mM to about 10 mM, from about 1 mM to about 5 mM. In a very specific embodiment, the concentration of monosodium phosphate in the pRBC storage solution is about 4 mM.

In embodiments, the solutions disclosed herein may comprise a concentration of sodium chloride (NaCl) of from about 0 mM to about 100 mM, from about 1 mM to about 100 mM, from about 10 mM to about 90 mM, from about 10 mM to about 80 mM, from about 20 mM to about 80 mM, from about 30 mM to about 80 mM, from about 40 mM to about 80 mM, from about 50 mM to about 80 mM, from about 60 mM to about 80 mM, or about 70 mM.

In a specific embodiment, the solution is a pRBC storage solution and the concentration of sodium chloride (NaCl) ranges from about 25 mM to about 100 mM, from about 50 mM to about 100 mM, from about 60 mM to about 100 mM, from about 60 mM to about 90 mM, or from about 60 mM to about 80 mM. In a very specific embodiment, the concentration of sodium chloride in the pRBC storage solution is about 70 mM.

In a specific embodiment, the solutions disclosed herein may comprise a concentration of sodium bicarbonate (NaHCO₃) of from about 0 mM to about 30 mM, from about 1 mM to about 30 mM, from about 1 mM to about 25 mM, from about 1 mM to about 20 mM, from about 1 mM to about 15 mM, from about 1 mM to about 10 mM, from about 1 mM to about 5 mM, from about 5 mM to about 30 mM, from about 10 mM to about 30 mM, from about 15 mM to about 30 mM, from about 20 mM to about 30 mM.

In embodiments, the presently disclosed solutions may further comprise one or more nucleosides, illustratively selected from adenine, guanosine, and combinations thereof. In embodiments, the disclosed solutions may comprise a concentration of one or more nucleosides that ranges from about 0 mM to about 10 mM, from about 1 mM to about 10 mM, from about 1 mM to about 9 mM, from about 1 mM to about 8 mM, from about 1 mM to about 7 mM, from about 1 mM to about 6 mM, from about 1 mM to about 5 mM, from about 1 mM to about 4 mM, from about 1 mM to about 5 mM, from about 1 mM to about 4 mM, from about 1 mM to about 3 mM, from about 1 mM to about 2 mM, from about 2 mM to about 3 mM, from about 2 mM to about 4 mM, from about 2 mM to about 5 mM, about 1 mM, about 2 mM, about 3 mM, about 4 mM, about 5 mM, about 6 mM, about 7 mM, or about 8 mM. In a specific embodiment, the solution is a pRBC storage solution and the concentration of nucleoside is from about 1 mM to about 5 mM. In a very specific embodiment, the pRBC storage solution comprises about 2.22 mM adenine.

In embodiments, the presently disclosed solutions may further comprise one or more sugars, illustratively selected from the group consisting of dextrose, glucose, and combinations thereof. In embodiments, the disclosed solutions may comprise a concentration of sugar of from about 1 mM to about 500 mM, from about 10 mM to about 500 mM, from about 20 mM to about 500 mM, from about 30 mM to about 500 mM, from about 40 mM to about 500 mM, from about 50 mM to about 500 mM, from about 50 mM to about 400 mM, from about 50 mM to about 300 mM, from about 50 mM to about 275 mM, from about 50 mM to about 260 mM, from about 50 mM to about 250 mM, from about 50 mM to about 200 mM, from about 50 mM to about 150 mM, from about 50 mM to about 100 mM, from about 50 mM to about 90 mM, from about 50 mM to about 80 mM, from about 50 mM to about 70 mM, or from about 50 mM to about 60 mM.

In a specific embodiment, the solution is an anticoagulant collection solution and the concentration of sugar is from about 100 mM to about 300 mM, from about 150 mM to about 300 mM, from about 200 mM to about 300 mM, from about 250 mM to about 300 mM, or from about 250 mM to about 275 mM. In a specific embodiment, the anticoagulant collection solution comprises from about 250 mM to about 275 mM dextrose. In a very specific embodiment, the anticoagulant collection solution comprises about 257 mM dextrose.

In another specific embodiment, the solution is a pRBC storage solution and the concentration of sugar is from about 1 mM to about 100 mM, from about 10 mM to about 100 mM, from about 10 mM to about 90 mM, from about 10 mM to about 80 mM, from about 10 mM to about 70 mM, from about 20 mM to about 70 mM, from about 30 mM to about 70 mM, from about 40 mM to about 70 mM, from about 40 mM to about 60 mM, or from about 50 mM to about 60 mM. In a specific embodiment, the pRBC storage solution comprises from about 40 to about 60 mM dextrose. In a very specific embodiment, the pRBC storage solution comprises about 56 mM dextrose.

In embodiments, the presently disclosed solutions may further comprise one or more membrane protectants, illustratively selected from the group consisting of citrate, citric acid, mannitol, and combinations thereof.

Osmolarity refers to the solute concentration as defined by the number of osmoles of solute per liter of solution (Osm/L). In embodiments the disclosed solutions comprise an osmolarity that ranges from about 250 Osm/L to about 700 Osm/L, from about 300 Osm/L to about 700 Osm/L, from about 320 Osm/L to about 700 Osm/L, from about 320 Osm/L to about 600 Osm/L, from about 320 Osm/L to about 500 Osm/L, from about 320 Osm/L to about 400 Osm/L, or from about 320 Osm/L to about 350 Osm/L.

In a specific embodiment, the solution is an anticoagulant collection solution and the osmolarity ranges from about 600 Osm/L to about 700 Osm/L, from about 625 Osm/L to about 700 Osm/L, from about 650 Osm/L to about 675 Osm/L, or from about 660 Osm/L to about 675 Osm/L. In a very specific embodiment, the anticoagulant collection solution comprises an osmolarity of about 670 mM.

In another specific embodiment, the solution is a pRBC storage solution and the osmolarity ranges from about 250 Osm/L to about 400 Osm/L, from about 300 Osm/L to about 350 Osm/L, from about 325 Osm/L to about 350 Osm/L, or from about 325 Osm/L to about 340 Osm/L. In a very specific embodiment, the pRBC storage solution comprises an osmolarity of about 326 mM.

pH of the solutions disclosed herein may range from about 5 to about 10, about 5.5 to about 10, about 6 to about 10, about 6.5 to about 10, about 7 to about 10, about 7.5 to about 10, about 8 to about 10, about 8 to about 9.5, or about 8 to about 9. In embodiments, the pH of the solutions disclosed herein is about 5, about 6, about 7, about 8, about 9, or about 10.

In a specific embodiment, the solution is an anticoagulant collection solution and the pH ranges from about 8 to about 10. In a very specific embodiment, the pH of the anticoagulant collection solution is about 8. In another very specific embodiment, the pH of the anticoagulant solution is about 8.4.

In another specific embodiment, the solution is a pRBC storage solution and the pH ranges from about 8 to about 10. In a very specific embodiment, the pH of the anticoagulant collection solution is about 8. In another very specific embodiment, the pH of the anticoagulant collection solution is about 8.1.

In some embodiments, the solutions disclosed herein are substantially free of citrate and/or citric acid.

Whole blood is collected in vessels comprising anticoagulant solutions to prevent clotting and avoid changes in metabolic and therapeutic qualities of blood. Standard anticoagulant collection solutions include, for example, calcium phosphate double dextrose (CP2D), citrate phosphate dextrose adenine (CPDA), citrate phosphate dextrose (CPD), additive solutions, and the like. Such solutions are known in the art and available from a variety of vendors. Generally, whole blood or pRBCs may be stored in anticoagulant solutions for a limited period of time, i.e., up to 21 days for CPD or CP2D, and up to 35 days for CPDA. Additive solutions comprise additional dextrose and adenine, permitting longer storage of RBCs, i.e., up to 42 days.

Various additive solutions are known in the art, illustratively including Additive Solution-1 (AS-1, available commercially as Adsol®), Additive Solution-3 (AS-3, available commercially as Nutricel®), Additive Solution-5 (AS-5, available commercially as Optisol®), Additive Solution-7 (AS-7, available commercially as SOLX®), saline-adenine-glucose-mannitol solution (SAGM), phosphate-adenine-glucose-guanosine-saline-mannitol solution (PAGGSM), phosphate-adenine-glucose-guanosine-gluconate-mannitol (PAG3M), Erythro-Sol (E-Sol), E-Sol 5, and the like. Formulations and guidelines for making for suitable additive solutions are known in the art. Commercial additive solutions are available from a variety of vendors.

Solutions according to the present disclosure may be formulated by adding one or more inorganic pyrophosphates to a pre-made anticoagulant collection solution or additive solution. For example, in embodiments, solutions according to the present disclosure comprise a solution selected from the group consisting of AS-1, AS-3, AS-5, AS-7, SAGM, PAGGSM, PAG3M, E-Sol, E-Sol-5, CP2D, CPd, CPDa, and the like. Such solutions may be modified by adding one or more inorganic pyrophosphates (PPi) to provide an anticoagulant collection or storage solution according to the present disclosure.

In certain embodiments, a kit is provided, the kit comprising: (1) an anticoagulant blood collection solution and/or pRBC storage solution, and (2) one or more inorganic pyrophosphates, optionally in the form of a solution. In embodiments, the kit comprises (1) one or more of a solution selected from the group consisting of AS-1, AS-3, AS-5, AS-7, SAGM, PAGGSM, PAG3M, E-Sol, E-Sol-5, CP2D, CPd, and CPDa; and (2) one or more inorganic pyrophosphates, optionally in the form of a solution, as disclosed herein.

In certain kit embodiments, the solutions are each separately packaged, for example, in bags, vials, tubes, or combinations thereof. In embodiments, the kit further comprises instructions for use.

In another embodiment, a method of preserving red blood cells is provided, the method comprising collecting and/or storing whole blood and/or pRBCs in a solution comprising one or more inorganic pyrophosphates as disclosed herein.

In another embodiment, a method for mitigating a complication associated with a transfusion or infusion of whole blood or red blood cells is provided, the method comprising collecting and/or storing the red blood cells in a solution comprising one or more inorganic pyrophosphates as disclosed herein and, optionally, administering the red blood cells to a subject in need thereof via transfusion or infusion, thereby mitigating the complication associated with transfusion or infusion of whole blood or red blood cells. In embodiments, administration of red blood cells (either in the form of whole blood, or pRBCs) collected and/or stored in the presently disclosed solutions mitigates or reduces the severity of or reduces the likelihood of the recipient subject experiencing a complication selected from the group consisting of transfusion-related acute lung injury (TRALI), pulmonary thrombosis, deep vein thrombosis, dyspnea, hypoxemia, hypotension, hypertension, fever, acute respiratory distress, acute respiratory distress syndrome (ARDS), systemic inflammatory response, organ failure, or death. In embodiments, the solution comprising red blood cells is suitable for direct infusion or transfusion into the subject, without the need for further washing or preparation.

### EXAMPLES

The following examples are provided by way of illustration and are in no way intended to limit the scope of the present disclosure.

### Example 1. Formulation of Anticoagulant Collection Solutions and pRBC Storage Solutions

Tetrasodium pyrophosphate was substituted for citric acid and sodium citrate in a standard citrate phosphate double dextrose anticoagulant solution (CP2D) to provide a novel anticoagulant collection solution, PPi double dextrose (PPi2D), according to concentrations as set forth in Table 1.

TABLE 1
CP2D and PPi2D Formulations (mMol)

| Component | CP2D | PP12D |
|---|---|---|
| Na₂ Citrate | 89 | — |
| Citric Acid | 17 | — |
| NaH₂PO₄ | 19 | 112 |
| NaCl | — | — |
| Adenine | — | — |
| Dextrose | 257 | 257 |
| Na₄PPᵢ | — | 37 |
| Osmolarity | 670 | 670 |
| pH | 5.7 | 8.4 |

Tetrasodium pyrophosphate was substituted for citric acid and sodium citrate in a standard Additive Solution-3 (AS-3) to provide a novel pRBC storage solution, PPi-3, according to concentrations as set forth in Table 2.

TABLE 2
AS-3 and PPi-3 Formulations (mMol)

| Component | AS-3 | PPi-3 |
|---|---|---|
| Na₂ Citrate | 20 | — |
| Citric Acid | 2 | — |
| NaH₂PO₄ | 23 | 4 |
| NaCl | 70 | 70 |
| Adenine | 2.22 | 2.22 |
| Dextrose | 56 | 56 |
| Na₄PPᵢ | — | 24 |
| Osmolarity | 326 | 326 |
| pH | 5.6 | 8.1 |

### Example 2. Collection of Whole Human Blood in PPi2D and Storage of Human pRBCs in PPi-3 Yields Improved RBC Quality Compared to CP2D and AS-3, Respectively

Human whole blood was obtained from seven healthy volunteers via routine phlebotomy techniques according to a protocol approved by the University of Cincinnati Institutional Review Board. The whole blood sample from each donor was split and placed in either the anticoagulant citrate phosphate double dextrose (CP2D) or the disclosed anticoagulation pyrophosphate double dextrose (PPi2D) of Table 1. The banked whole blood underwent centrifugation at 300 g for 7 minutes for which the platelet rich plasma was discarded. Subsequently the blood underwent centrifugation at 1,000 g for 15 minutes. The platelet poor plasma and buffy coat containing leukocytes was aspirated and discarded. The erythrocyte pellet was resuspended in AS-3 or the disclosed PPi-3 preservative solution as set forth in Table 2 in a 2:9 ratio, and stored for the Food and Drug Administration (FDA) approved storage duration of 42 days at 4° C.

At the end of the storage duration, human pRBCs units were analyzed for microvesicle accumulation, band-3 membrane integrity protein expression, phosphatidylserine expression, erythrocyte viability, the presence of oxidative stress via advanced oxidative protein products (AOPP), free hemoglobin release, and susceptibility to osmotic stress.

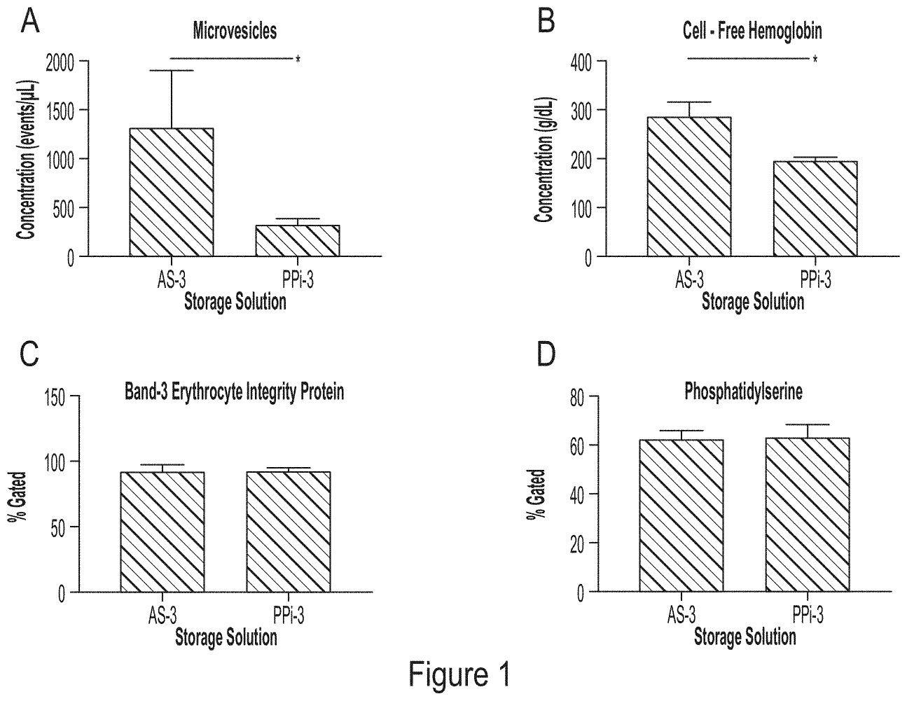

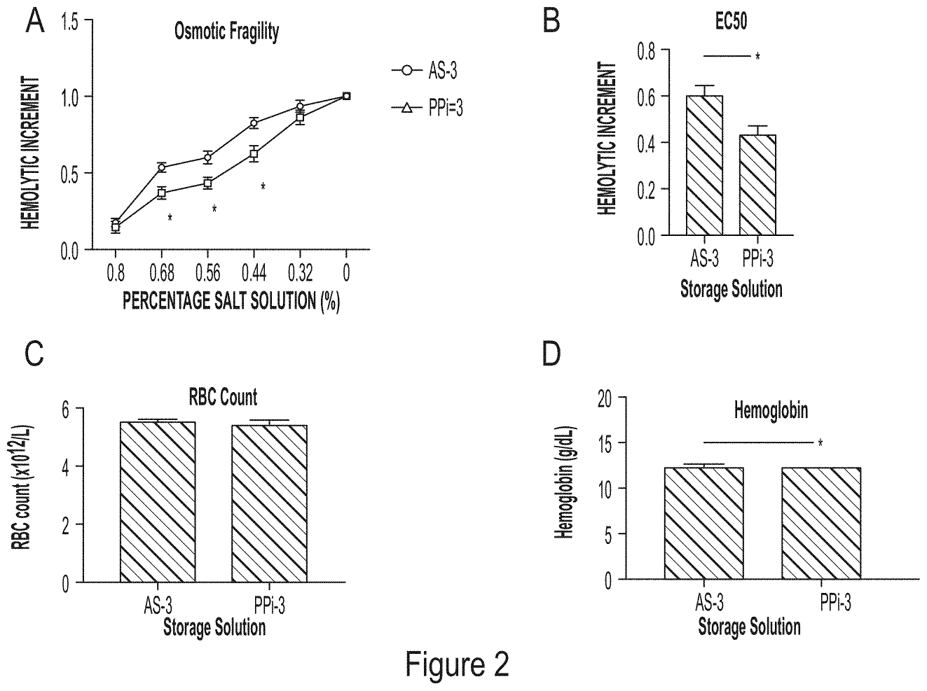

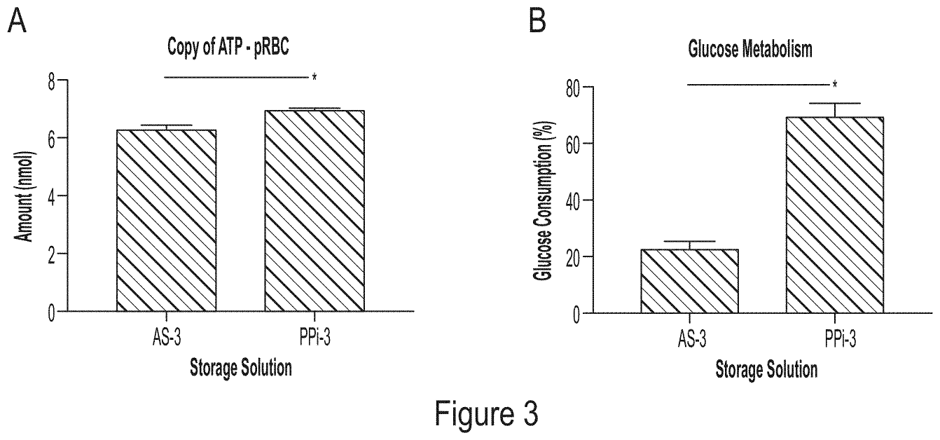

As compared to AS-3 storage solution, use of the PPi-3 storage solution over the 42-day storage duration of human pRBCs resulted in a reduction in microvesicle (MV) and cell-free hemoglobin release (FIG. 1A-B). There was no difference in Band-3 erythrocyte membrane integrity protein or phosphatidylserine expression (FIG. 1C-D). Red blood cells stored in PPi-3 demonstrated a reduction in susceptibility to osmotic stress, with reduced EC50 (FIG. 2A-B). Reduced presence of AOPP as compared to AS-3 was also observed (data not shown). There was no difference in red blood cell count, and increased intracellular hemoglobin content was observed (FIG. 2C-D). There was an increase in the presence of ATP (FIG. 3A) as well as increased glucose metabolism compared to cells in standard AS-3 (FIG. 3B). These results indicate collection and storage of pRBCs in the PPi2D and PPi-3 solutions disclosed herein provide marked a marked improvement in RBC quality compared to standard CP2D and AS-3 solutions.

### Example 3. Storage of Murine pRBCs in PPi-3 Compared to AS-3 Yields Improved RBC Quality

Murine experiments were performed in accordance with the Institutional Animal Care and Use Committee of the University of Cincinnati. Male 8-10 week old C57BL/6 mice obtained from Jackson Laboratory (Bar Harbor, Me.) were acclimated for 2 weeks in a climate controlled room with a 12 hour light dark cycle and fed with standard pellet diet and water ad libitum. Murine whole blood banking was performed using a modification of our previously characterized protocol (Makley, et al., Murine Blood Banking: Characterization and Comparisons to Human Blood, Shock 34(1): 40-45 (2010)). The mice were anesthetized with intraperitoneal pentobarbital (0.1 mg/gram body weight) and whole blood obtained via cardiac puncture. Packed red blood cell units were generated via density gradient centrifugation at 400 g for 40 minutes with subsequent resuspension of the erythrocytes in AS-3 or PPi-3 storage solution in a 2:9 ratio and storage for up to 14 days. It has previously been shown that 14 days of storage for murine pRBCs is equivalent to 42 days of pRBC storage in human units.

### Red Blood Cell Storage Lesion

At the end of the storage duration, murine pRBCs units were analyzed for microvesicle accumulation, band-3 membrane integrity protein expression, phosphatidylserine expression, erythrocyte viability, the presence of oxidative stress via advanced oxidative protein products (AOPP), free hemoglobin release, and susceptibility to osmotic stress.

Samples underwent centrifugation at 2,000 g for 10 minutes, followed by collection of supernatant and centrifugation at 10,000 g for 10 minutes. The supernatant was collected, with care taken to avoid the red blood cell pellet, then centrifuged at 21,100 g for 35 minutes in order to pellet the microvesicles. Phycoerythrin (PE) Rat, anti-mouse, Ter 119 antibody (mouse) and CD235a (human; BD Biosciences San Jose, Calif.) was utilized to quantify RBC-derived microvesicles on an Attune Flow Cytometer (Life Technologies, Foster City, Calif.).

To determine the percentage of RBCs expressing Band-3 membrane integrity protein (Band-3) and phosphatidylserine on the erythrocyte surface, the antibody Eosin-5-malemeide (EMA) and Fluorescein isothiocyanate (FITC) Annexin V (BD Biosciences San Jose, Calif.), respectively, were utilized and quantified via flow cytometry. Cell-free hemoglobin concentration was measured via hemoglobin colorimetric assay (BioVision Inc., Milpitas, Calif.) read at 575 nm on microplate spectrophotometer (BioTek Cytation 5, Winooski, Vt.). Oxidative stress was calculated via AOPP concentrations (Cell Biolabs Inc., San Diego, Calif.) read at 340 nm on the microplate spectrophotometer. Susceptibility of red blood cells to osmotic stress was determined by suspending aliquots of erythrocytes in solutions containing increasing concentrations of sodium chloride (0, 0.32, 0.44, 0.56, 0.68, and 0.8% NaCl) for 30 minutes, followed by centrifugation at 10,000×g for 10 minutes with analysis of the supernatant absorbance measured via a microplate spectrophotometer at 575 nm. The hemolytic increment was calculated and EC50 determined by the hemolytic increment of each sample when suspended in the 0.56% NaCl solution.

### Erythrocyte Structural Evaluation

Peripheral smears were evaluated for any visible structural changes that the erythrocytes underwent during storage. In order to confirm the structural findings from the peripheral smear, a forward scatter and side scatter evaluation was performed via flow cytometry.

### Erythrocyte Biochemical Evaluation

The red blood cell content was measured on a Coulter Ac·T diff Hematology Analyzer (Beckman Coulter, Brea, Calif.). i-STAT handheld blood analyzer (Abbott Laboratories, Chicago, Ill.) was utilized to obtain blood gas, electrolyte, and hematologic information. After the erythrocytes were lysed by combining 5 μl pRBCs with 200 μl of 1× cell lysis buffer (BioVision Inc., Milpitas, Calif.) an ATP Colorimetric Assay (BioVision Inc., Milpitas, Calif.) was utilized to quantify intracellular ATP via a microplate spectrophotometer.

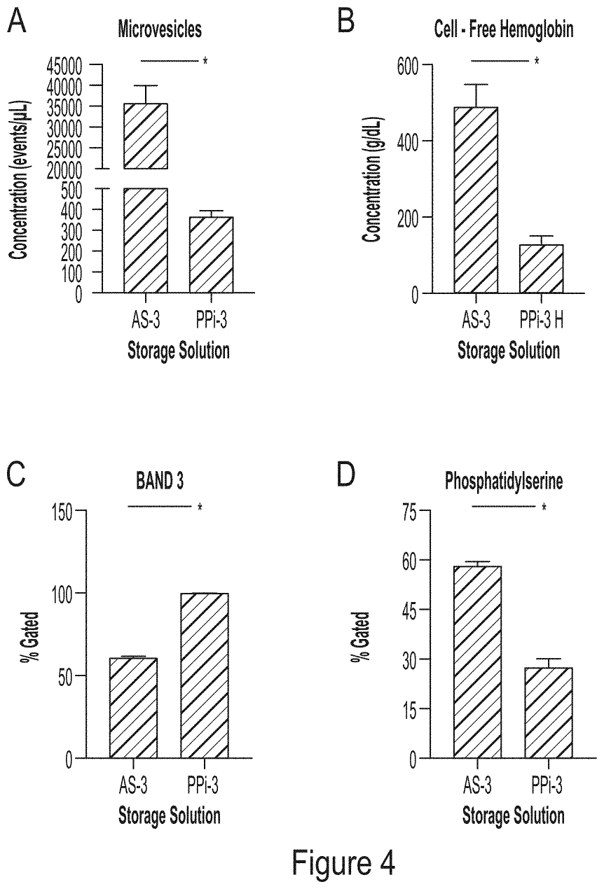

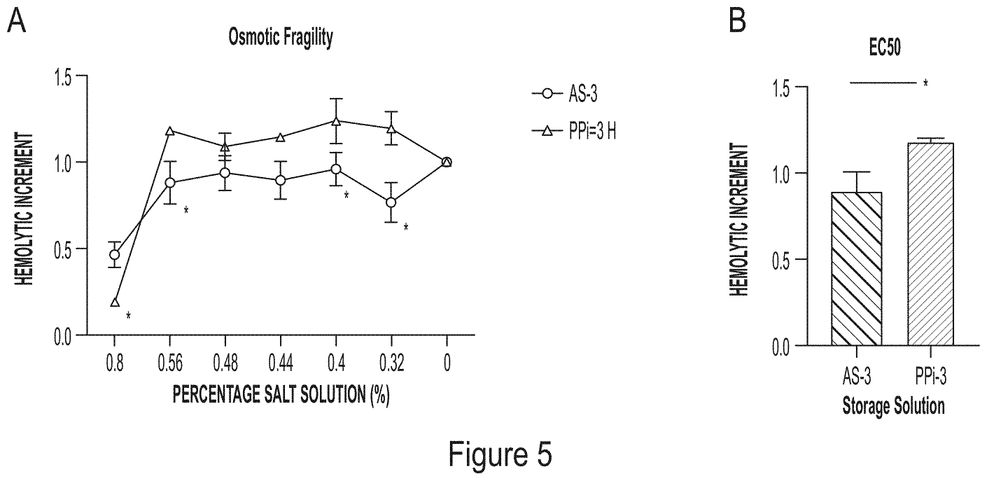

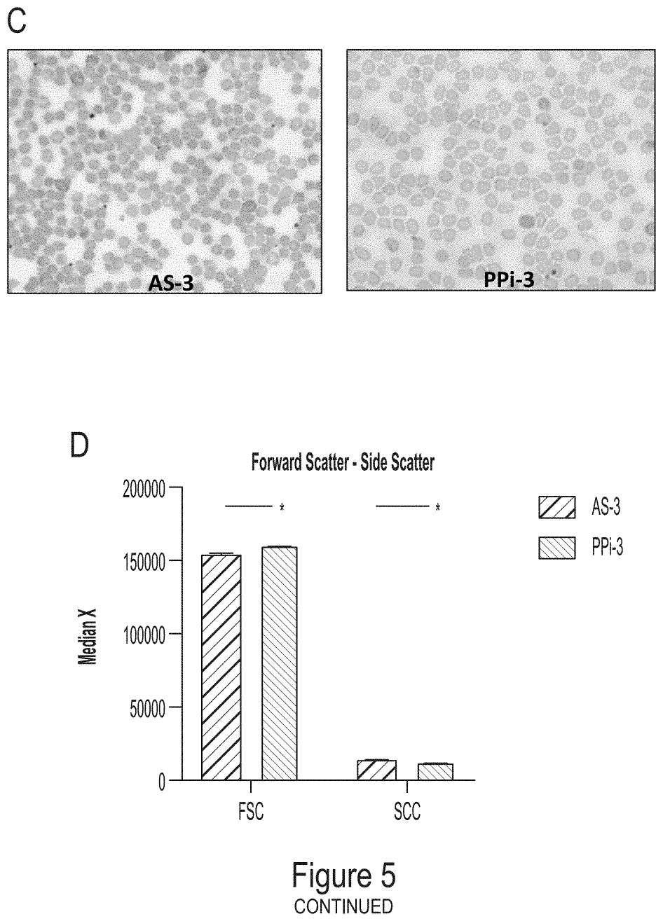

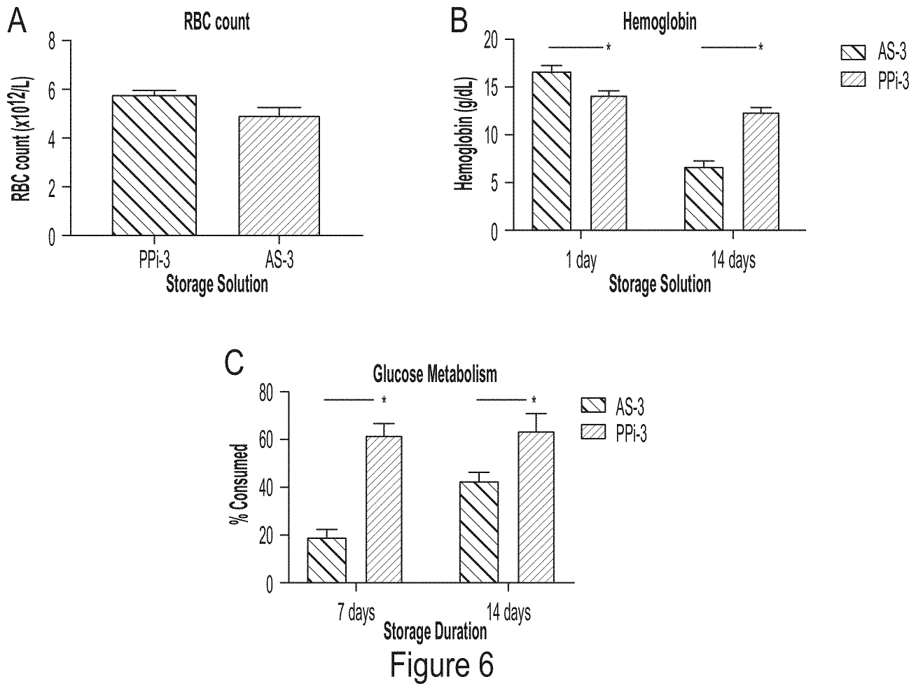

Results showed a reduction in microvesicle accumulation and free hemoglobin concentration in the RBC milieu (FIG. 4A-B). There was maintenance of Band-3 expression as well as reduced phosphatidylserine expression of RBCs stored in PPi-3 (FIG. 4C-D). However, compared to AS-3 there was a slight increase in osmotic fragility with increased EC50 (FIG. 5A-B). On peripheral smear analysis, the erythrocytes stored in PPi-3 appeared to be greater in size, as well as with less membrane complexity, as defined by the presence of RBC spherocytosis (FIG. 5C). The PPi-3 RBC had significantly greater forward scatter (FSC) and less side scatter (SCC) on flow cytometry (FIG. 5D). Similar to human studies, there was no difference in red blood cell count but there was a significantly greater intracellular hemoglobin content and greater glucose metabolism (FIG. 6A-C). These results indicate storage of pRBCs in the PPi-3 solution disclosed herein provides marked a marked improvement in RBC quality compared to standard CP2D and AS-3 solutions.

### Example 4. Mice Resuscitated with pRBCs Stored in PPi-3 Demonstrate Greater Hemodynamic Response Compared to pRBCs Stored in AS-3

Male C57BL/6 mice were anesthetized with intraperitoneal pentobarbital (0.1 mg/gram body weight) followed by groin clipping and sterile preparation with povidone-iodine solution and alcohol. The skin was incised, femoral vessels exposed, and the femoral artery cannulated with a tapered polyethylene catheter. The catheter was connected to pressure transducers for continuous hemodynamic monitoring of the mice (AD Instruments Lab Chart). To avoid hypothermia, the cannulated mice were placed on a circulating water blanket maintained at 41° C. After 10 minutes of equilibration, hemorrhagic shock was obtained by withdrawing blood to achieve a mean arterial pressure (MAP) of 25+5 mmHg and maintained for 60 minutes. The volume (mL) of blood required to achieve the desired hemorrhagic shock MAP was recorded for each mouse. Following hemorrhagic shock, mice were resuscitated with pRBCs stored in either AS-3 or PPi-3 storage solution to achieve a MAP greater than 70 mm Hg±5 mm Hg. The volume (mL) of fluid or blood required to achieve the appropriate resuscitation was recorded for each resuscitation group. The mice were monitored for 15 minutes following resuscitation, femoral artery decannulated, and euthanized at 1 hour post procedure end. Sham animals underwent femoral artery cannulation and hemodynamic monitoring for 90 minutes, without hemorrhage or resuscitation.

One hour after hemorrhage and resuscitation, mice were sacrificed, and whole blood obtained in a serum separator tube (SST). Following 30 minutes the SST underwent centrifugation at 8,000 rpm for 10 minutes in order to isolate the serum. Serum samples were analyzed for inflammatory chemokines and cytokines utilizing a flow cytometry-based cytometric bead array assay (BD Biosciences, San Jose, Calif.).

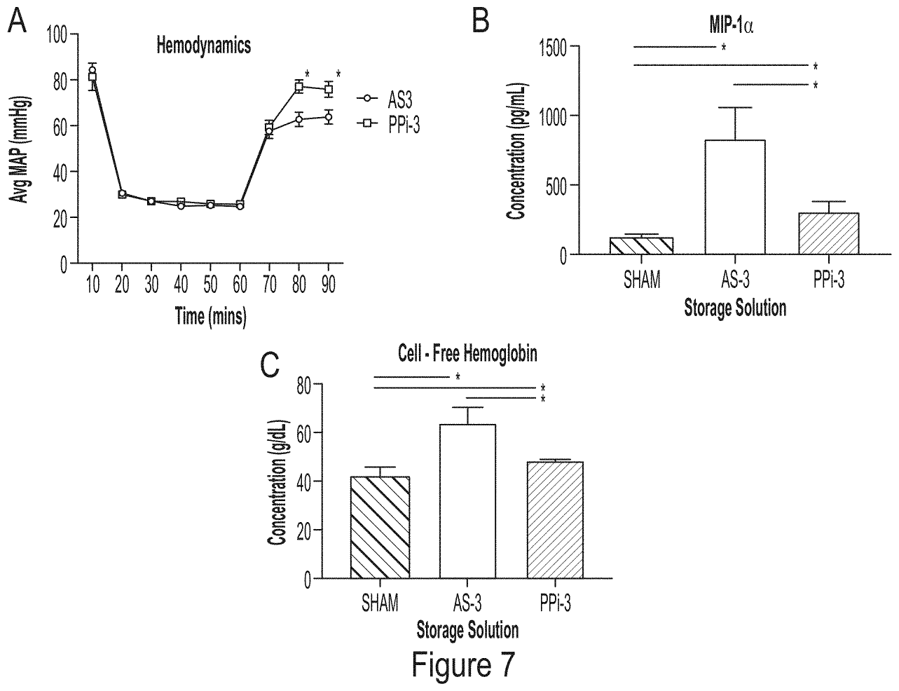

After hemorrhage, mice resuscitated with pRBCs stored in PPi-3 storage solution demonstrated a greater hemodynamic response to resuscitation than the AS-3 group (FIG. 7A). Analysis of serum from the recipient mice demonstrated a significant attenuation in the inflammatory marker macrophage inflammatory protein-1-alpha (MIP-1 α; FIG. 7B) as well as reduced presence of cell-free hemoglobin in the serum (FIG. 7C). There was no difference in the volume of pRBCs transfused during resuscitation. These results indicate resuscitation with pRBCs stored in PPi-3 reduces the risk of inflammatory response due to transfusion of aged pRBCs.

### Example 5. Mouse pRBCs Stored in PPi-3 Elicit Reduced Inflammatory Response in Vitro Compared to pRBCs Stored in AS-3

### Cultured Endothelial Cell Model

Isolated C57BL/6 mouse primary lung microvascular endothelial cells (MLEC) were obtained (Cell Biologics, Chicago, Ill.) and grown in mouse endothelial cell medium supplemented with endothelial cell growth supplement, antibiotics, and fetal bovine serum. MLEC cells were counted and plated in 24-well plates with an area of 1.9 cm²/well. We ensured that the wells were seeded at 5000 cells/cm² and grown to confluence prior to experimentation. The cells were treated with tumor necrosis factor alpha (0.02 μg/ml), pRBCs in AS-3, pRBCs in PPi-3, or media alone. Hematocrit of the pRBCs was determined via the Coulter Ac·T diff Hematology Analyzer and subsequently pRBCs were diluted in media in order to obtain a hematocrit of 5%. After a 24-hour treatment period in an incubator at 37° C., the supernatant was obtained and analyzed for inflammatory cytokines via a flow cytometry-based cytometric bead array (CBA) assay.

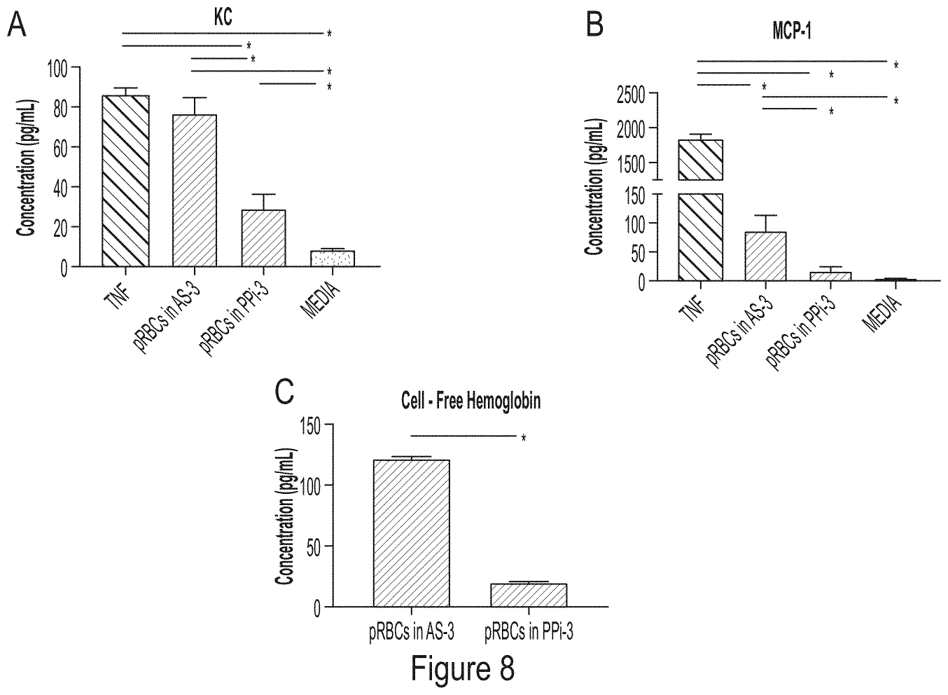

The effect of pRBCs stored in either AS-3 or PPi-3 on the inflammatory response in murine lung endothelial cells was assessed. Results showed that treatment of MLEC cells with pRBCs stored in AS-3 resulted in increased levels of the inflammatory cytokines keratinocyte chemoattractant (KC) and monocyte chemoattractant protein-1 (MCP-1) in the media (FIG. 8A-B). Treatment with pRBCs stored in PPi-3 resulted in an attenuated inflammatory response under these conditions (FIG. 8A-B). Reduced presence of cell-free hemoglobin was detected for PPi-3 compared to AS-3 (FIG. 8C). These results indicate storage of pRBCs in PPi-3 reduces the inflammatory response compared to pRBCs stored in AS-3.

### Example 6. Exemplary Formulations of pRBC Storage Solutions

Tetrasodium pyrophosphate was substituted for citric acid and sodium citrate in a standard Additive Solution-3 (AS-3) to provide two exemplary pRBC storage solutions, PPi-3_{L} and PPI-3_{H}, according to concentrations as set forth in Table 3. Low (L) and high (H) concentrations of components were assessed.

TABLE 3
Low (PPi-3_{L}) and high (PPI-3_{H}) exemplary PPi-3
formulations and AS-3 (mMol)

| Component | AS-3 | PPi-3_{L} | PPi-3o |
|---|---|---|---|
| Na₂ Citrate | 20 | — | — |
| Citric Acid | 2 | — | — |
| NaH₂PO₄ | 23 | 23.00 | 23.00 |
| NaCl | 70 | 70.16 | 45.79 |
| Adenine | 2.22 | 2.22 | 2.22 |
| Dextrose | 56 | 50.46 | 50.46 |
| Na₄PPᵢ | — | 12.43 | 22.18 |
| Osmolality | 326 | 300 | 300 |
| ImOsm/kg) | | | |
| pH | 5.6 | 6.7 | 7.3 |

### Example 7. Use of PPi-3 pRBC Storage Solution Results in Decreased Microparticle Accumulation Compared to AS-3 During the Storage Period

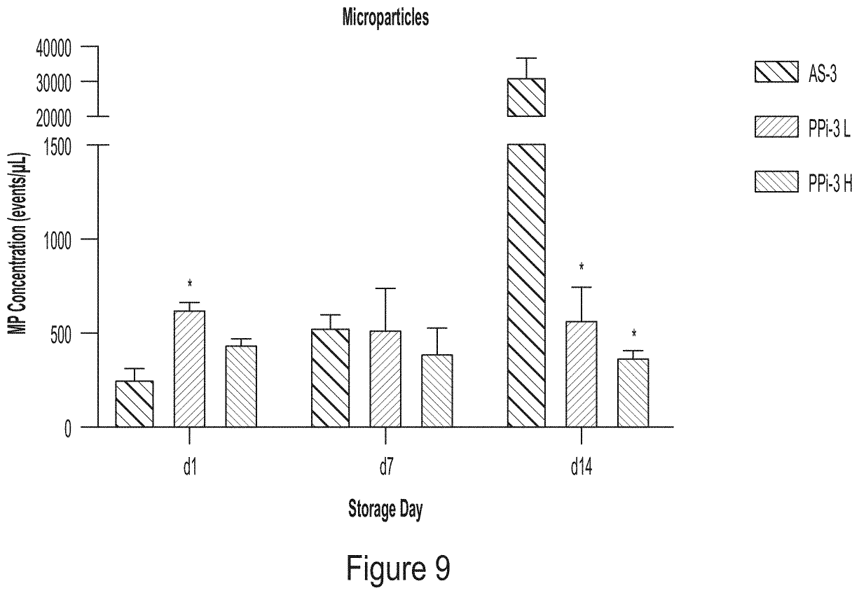

Murine pRBCs were collected and stored as described above in each of AS-3, PPi-3_{L}, and PPi-3_{H} storage solutions. Samples were assayed for microparticle concentration at day 1, day 7, and day 14. As shown in FIG. 9, decreased microparticle accumulation was observed in each of PPi-3_{L} and PPi-3_{H} compared to AS-3 at the end of the storage period.

### Example 8. Use of PPi-3 pRBC Storage Solution Results in Preserved Erythrocyte Band 3 During the Storage Period

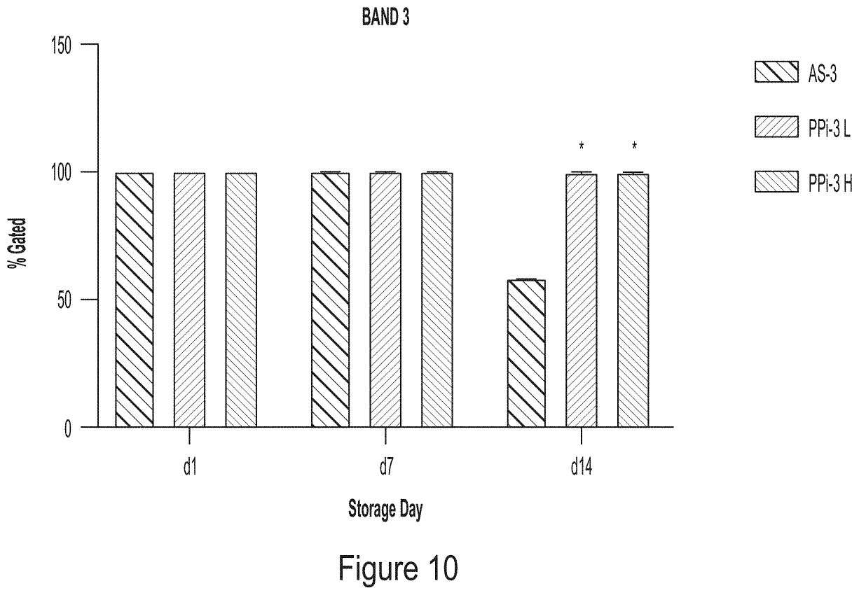

Murine RBCs were collected and stored as pRBCs as described above in each of AS-3, PPi-3_{L}, and PPi-3_{H} storage solutions. pRBCs units were analyzed for Band-3 membrane integrity protein expression at day 1, day 7, and day 14 of storage. As shown in FIG. 10, erythrocyte Band 3 protein expression was preserved for each of PPi-3_{L} and PPi-3_{H} compared to AS-3 across the storage period.

### Example 9. Use of PPi-3 pRBC Storage Solution Results in Decreased Annexin V Expression During the Storage Period

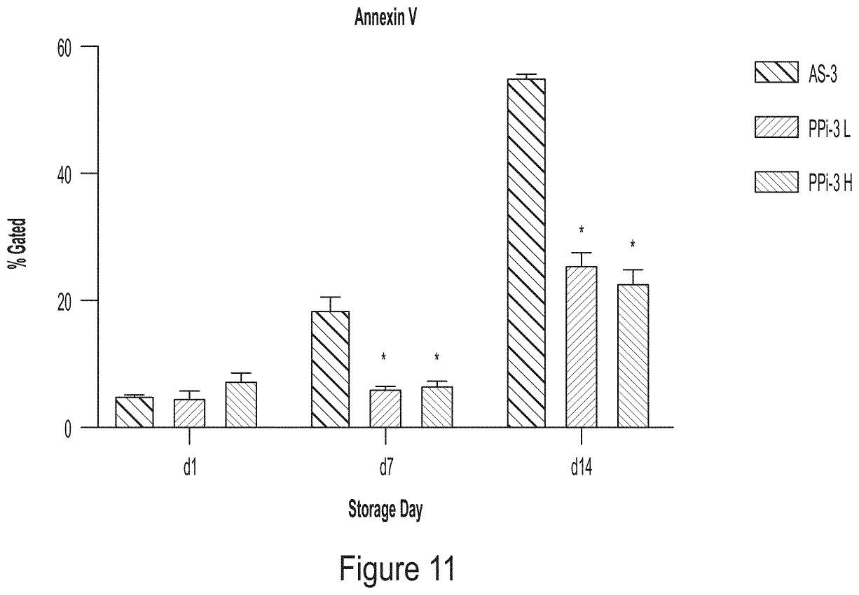

Murine RBCs were collected and stored as pRBCs as described above in each of AS-3, PPi-3_{L}, and PPi-3_{H} storage solutions. pRBCs units were analyzed for Annexin V expression at day 1, day 7, and day 14 of storage. As shown in FIG. 11, Annexin V expression was decreased for each of PPi-3_{L} and PPi-3_{H} compared to AS-3 at days 7 and 14 of the storage period.

### Example 10. Use of PPi-3 pRBC Storage Solution Results in Decreased Advanced Oxidative Protein Products During the Storage Period

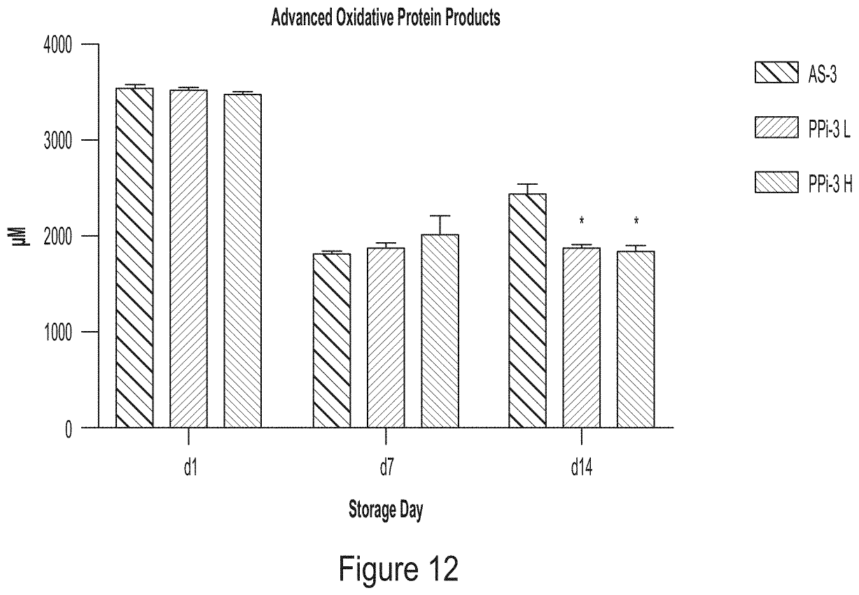

Murine RBCs were collected and stored as pRBCs as described above in each of AS-3, PPi-3_{L}, and PPi-3_{H} storage solutions. pRBCs units were analyzed for advanced oxidative protein products (AOPP) at day 1, day 7, and day 14 of storage. As shown in FIG. 12, advanced oxidative protein product concentration was decreased for each of PPi-3_{L} and PPi-3_{H} compared to AS-3 at day 14 of the storage period.

### Example 11. Use of PPi-3 pRBC Storage Solution Results in Decreased Supernatant Free Hemoglobin Accumulation During the Storage Period

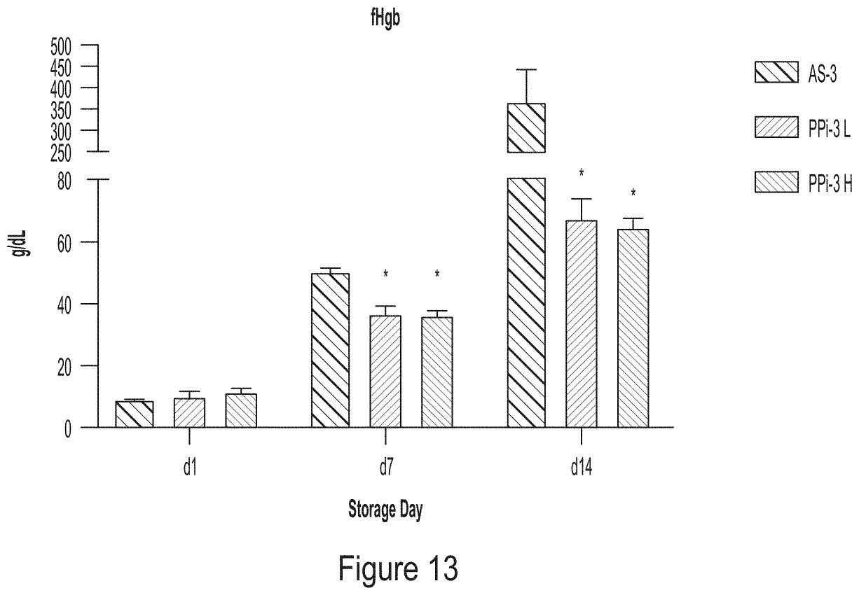

Murine RBCs were collected and stored as pRBCs as described above in each of AS-3, PPi-3_{L}, and PPi-3_{H} storage solutions. pRBCs units were analyzed for free supernatant hemoglobin at day 1, day 7, and day 14 of storage. As shown in FIG. 13, free hemoglobin concentration in the supernatant was decreased for each of PPi-3_{L} and PPi-3_{H} compared to AS-3 at days 7 and 14 of the storage period.

### Example 12. Use of PPi-3 pRBC Storage Solution Results in Increased pRBC Erythrocyte Hemoglobin During the Storage Period

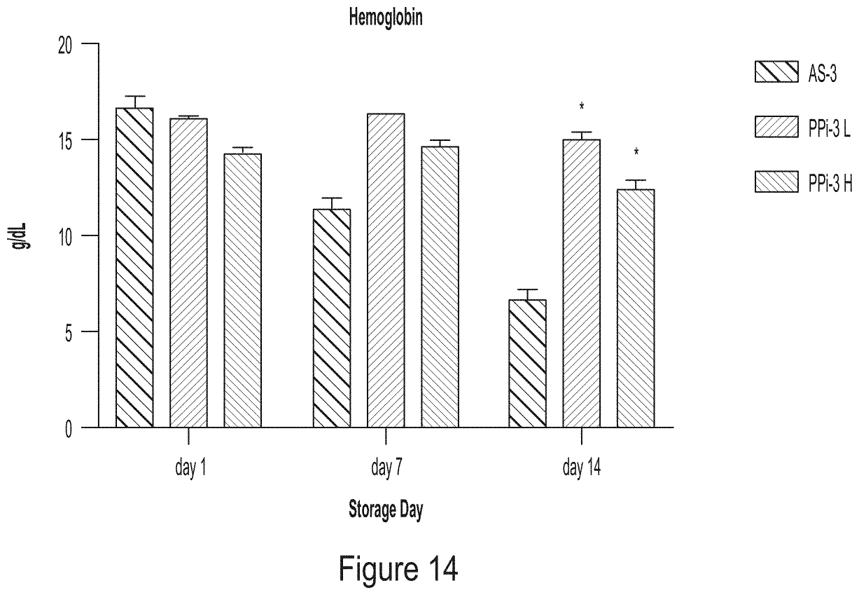

Murine RBCs were collected and stored as pRBCs as described above in each of AS-3, PPi-3_{L}, and PPi-3_{H} storage solutions. pRBCs units were analyzed for erythrocyte hemoglobin concentration at day 1, day 7, and day 14 of storage. As shown in FIG. 14, erythrocyte hemoglobin concentration was increased for each of PPi-3_{L} and PPi-3_{H} compared to AS-3 across the storage period.

### Example 13. Use of PPi-3 pRBC Storage Solution Results in Improved Base Deficit During the Storage Period

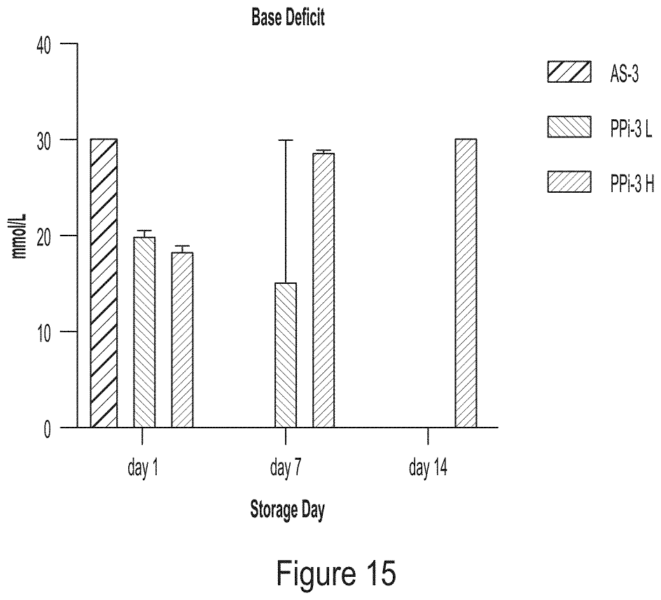

Murine RBCs were collected and stored as pRBCs as described above in each of AS-3, PPi-3_{L}, and PPi-3_{H} storage solutions. pRBCs units were analyzed for base deficit utilizing blood gas analysis at day 1, day 7, and day 14 of storage. As shown in FIG. 15, base deficit was improved for each of PPi-3_{L} and PPi-3_{H} compared to AS-3 through day 7, with superior performance by PPi-3_{H} storage solution at day 14.

### Example 14. Use of PPi-3 pRBC Storage Solution Results in Preservation of RBC Architecture During the Storage Period

Murine RBCs were collected and stored as pRBCs as described above in each of AS-3, PPi-3_{L}, and PPi-3_{H} storage solutions. pRBCs units were analyzed for RBC morphology at day 14 of storage. Peripheral smears were evaluated for any visible structural changes that the erythrocytes underwent during storage. In order to confirm the structural findings from the peripheral smear, a forward scatter and side scatter evaluation was performed via flow cytometry.

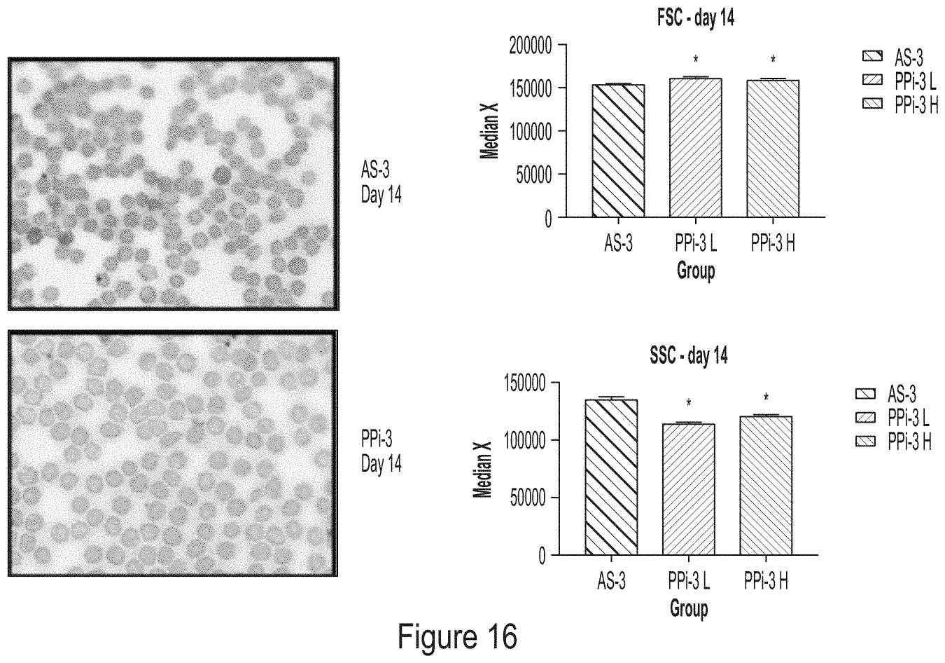

As shown in FIG. 16, RBC morphology was preserved for each of PPi-3_{L} and PPi-3_{H} compared to AS-3 at the end of the storage period. Left panel shows preserved RBC morphology via peripheral smear of PPi-3 compared to AS-3 (AS-3 top, PPi-3_{H}, bottom). Right panel shows forward scatter (top) and side scatter (bottom) for each of PPi-3_{L}, PPi-3_{H}, and AS-3 at day 14.

### Example 15. Exemplary Formulations of Collection Anticoagulant Solutions Comprising PPi

Tetrasodium pyrophosphate was substituted for citric acid and sodium citrate in a standard citrate phosphate double dextrose anticoagulant solution (CP2D) to provide two exemplary pRBC storage solutions, PPi2D_{H} (high) and PPi2D_{L} (low), according to concentrations as set forth in Table 4.

TABLE 4
Low (PPi2D_{L}) and high (PPi2D_{H})
anticoagulant collection solutions (mMol)

| Component | CP2D | PPi2D_{H} | PPᵢ2D_{L} |
|---|---|---|---|
| Na₂ Citrate | 117 | — | — |
| Citric Acid | 14 | — | — |
| NaH₂PO₄ | 16 | 18.52 | 90.72 |
| Dextrose | 284 | 257.91 | 257.91 |
| Na₄PPᵢ | — | 57.06 | 28.53 |
| Osmolality | | 580 | 580 |
| ImOsm/kg) | | | |
| pH | 5.6 | 8.3 | 6.7 |

Embodiments can be described with reference to the following numbered clauses, with preferred features laid out in dependent clauses.

1. A packed red blood cell (pRBC) storage solution comprising one or more inorganic pyrophosphates.
2. The pRBC storage solution according to clause 1, wherein the one or more inorganic pyrophosphates are selected from the group consisting of: monosodium diphosphate, disodium diphosphate, trisodium diphosphate, and tetrasodium pyrophosphate.
3. The pRBC storage solution according to any of the preceding clauses, wherein the one or more inorganic pyrophosphates is tetrasodium pyrophosphate.
4. The pRBC storage solution according to any of the preceding clauses, further comprising one or more electrolytes.
5. The pRBC storage solution according to clause 4, wherein the one or more electrolytes are selected from the group consisting of sodium chloride, monosodium phosphate, sodium citrate, sodium phosphate, sodium bicarbonate, sodium carbonate, calcium chloride, and combinations thereof.
6. The pRBC storage solution according to any of the preceding clauses, further comprising adenine.
7. The pRBC storage solution according to any of the preceding clauses, further comprising dextrose.
8. The pRBC storage solution according to any of the preceding clauses, further comprising an additive solution (AS).
9. The pRBC storage solution according to clause 8, wherein the additive solution is selected from the group consisting of Additive Solution-1 (AS-1), Additive Solution-3 (AS-3), Additive Solution-5 (AS-5), Additive Solution-7 (AS-7), and saline-adenine-glucose-mannitol solution (SAGM).
10. An anticoagulant whole blood collection solution comprising one or more inorganic pyrophosphates.
11. The anticoagulant collection solution according to clause 10, wherein the one or more inorganic pyrophosphates are selected from the group consisting of: monosodium diphosphate, disodium diphosphate, trisodium diphosphate, and tetrasodium pyrophosphate.
12. The anticoagulant collection solution according to any of clauses 10-11, wherein the one or more inorganic pyrophosphates is tetrasodium pyrophosphate.
13. The anticoagulant collection solution according to any of clauses 10-12, further comprising one or more electrolytes.
14. The anticoagulant collection solution according to clause 13, wherein the one or more electrolytes are selected from the group consisting of sodium chloride, monosodium phosphate, sodium citrate, sodium phosphate, sodium bicarbonate, sodium carbonate, calcium chloride, and combinations thereof.
15. The anticoagulant collection solution according to any of clauses 10-14, further comprising adenine.
16. The anticoagulant collection solution according to any of clauses 10-15, further comprising dextrose.
17. The anticoagulant collection solution according any of clauses 10-16, further comprising a solution selected from the group consisting of calcium phosphate double dextrose (CP2D), citrate phosphate dextrose adenine (CPDA), and citrate phosphate dextrose (CPD).
18. A method of preserving red blood cells, the method comprising storing whole blood or packed red blood cells (pRBCs) in a solution comprising one or more inorganic pyrophosphates.
19. The method according to clause 18, wherein the one or more inorganic pyrophosphates are selected from the group consisting of: monosodium diphosphate, disodium diphosphate, trisodium diphosphate, and tetrasodium pyrophosphate.
20. The method according to clause 18 or clause 19, wherein the solution further comprises:

one or more electrolytes,

adenine, and

dextrose.

21. The method according to any of clauses 18-20, wherein the solution further comprises an additive solution (AS).
22. The method according to clause 21, wherein the additive solution is selected from the group consisting of Additive Solution-1 (AS-1), Additive Solution-3 (AS-3), Additive Solution-5 (AS-5), Additive Solution-7 (AS-7), and saline-adenine-glucose-mannitol solution (SAGM).
23. The method according to clause 22, wherein the additive solution is AS-3.
24. The method according to clause 18, wherein the solution further comprises an anticoagulant solution selected from the group consisting of calcium phosphate double dextrose (CP2D), citrate phosphate dextrose adenine (CPDA), and citrate phosphate dextrose (CPD).
25. The method according to clause 24, wherein the anticoagulant solution is CP2D.
26. A method for mitigating a complication associated with a transfusion or infusion of red blood cells, the method comprising storing the red blood cells in a solution comprising one or more inorganic pyrophosphates.
27. The method according to clause 26, wherein the complication comprises one or more of transfusion-related acute lung injury (TRALI), pulmonary thrombosis, deep vein thrombosis, dyspnea, hypoxemia, hypotension, hypertension, fever, acute respiratory distress, acute respiratory distress syndrome (ARDS), systemic inflammatory response, organ failure, and death.
28. The method according clause 26 or clause 27, wherein the solution further comprises:

one or more electrolytes,

adenine, and

dextrose.

29. The method according to any of clauses 26-28, wherein the one or more inorganic pyrophosphates are selected from the group consisting of: monosodium diphosphate, disodium diphosphate, trisodium diphosphate, and tetrasodium pyrophosphate.
30. The method according to clause 26, wherein the solution further comprises an additive solution selected from the group consisting of Additive Solution-1 (AS-1), Additive Solution-3 (AS-3), Additive Solution-5 (AS-5), Additive Solution-7 (AS-7), and saline-adenine-glucose-mannitol solution (SAGM).
31. The method according to clause 26, wherein the solution further comprises an anticoagulant solution selected from the group consisting of calcium phosphate double dextrose (CP2D), citrate phosphate dextrose adenine (CPDA), and citrate phosphate dextrose (CPD).
32. A suspension of red blood cells comprising the composition according to any of clauses 1-17.
33. The suspension according to clause 32, wherein the suspension is suitable for direct infusion into a patient in need of such infusion.
34. The solution, method, or suspension according to any of the preceding clauses, wherein the one or more inorganic pyrophosphates is present at a total concentration of from about 1 mM to about 200 mM.
35. The solution, method, or suspension according to any of the preceding clauses, wherein the one or more inorganic pyrophosphates is tetrasodium pyrophosphate, and wherein the tetrasodium pyrophosphate is present at a concentration of from about 1 mM to about 200 mM.
36. The solution, method, or suspension according to any of the preceding clauses, wherein the one or more inorganic pyrophosphates is tetrasodium pyrophosphate, and wherein the tetrasodium pyrophosphate is present at a concentration of from about 5 mM to about 60 mM.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

We claim:

1. A packed red blood cell (pRBC) storage solution comprising one or more inorganic pyrophosphates.

2. The pRBC storage solution according to claim 1, wherein the one or more inorganic pyrophosphates are selected from the group consisting of: monosodium diphosphate, disodium diphosphate, trisodium diphosphate, and tetrasodium pyrophosphate.

3. The pRBC storage solution according to claim 2, wherein the one or more inorganic pyrophosphates is tetrasodium pyrophosphate.

4. The pRBC storage solution according to claim 1, further comprising one or more electrolytes.

5. The pRBC storage solution according to claim 4, wherein the one or more electrolytes are selected from the group consisting of sodium chloride, monosodium phosphate, sodium citrate, sodium phosphate, sodium bicarbonate, sodium carbonate, calcium chloride, and combinations thereof.

6. The pRBC storage solution according to claim 1, further comprising adenine.

7. The pRBC storage solution according to claim 1, further comprising dextrose.

8. The pRBC storage solution according to claim 1, further comprising an additive solution (AS).

9. The pRBC storage solution according to claim 8, wherein the additive solution is selected from the group consisting of Additive Solution-1 (AS-1), Additive Solution-3 (AS-3), Additive Solution-5 (AS-5), Additive Solution-7 (AS-7), and saline-adenine-glucose-mannitol solution (SAGM).

10-17. (canceled)

18. A method of preserving red blood cells, the method comprising storing whole blood or packed red blood cells (pRBCs) in a solution comprising one or more inorganic pyrophosphates.

19. The method according to claim 18, wherein the one or more inorganic pyrophosphates are selected from the group consisting of: monosodium diphosphate, disodium diphosphate, trisodium diphosphate, and tetrasodium pyrophosphate.

20. The method according to claim 18, wherein the solution further comprises:
one or more electrolytes,
adenine, and
dextrose.

21. The method according to claim 18, wherein the solution further comprises an additive solution (AS).

22. The method according to claim 21, wherein the additive solution is selected from the group consisting of Additive Solution-1 (AS-1), Additive Solution-3 (AS-3), Additive Solution-5 (AS-5), Additive Solution-7 (AS-7), and saline-adenine-glucose-mannitol solution (SAGM).

23. The method according to claim 22, wherein the additive solution is AS-3.

24. The method according to claim 18, wherein the solution further comprises an anticoagulant solution selected from the group consisting of calcium phosphate double dextrose (CP2D), citrate phosphate dextrose adenine (CPDA), and citrate phosphate dextrose (CPD).

25. The method according to claim 24, wherein the anticoagulant solution is CP2D.

26-31. (canceled)

32. A suspension of red blood cells comprising the composition according to claim 1.

33. The suspension according to claim 32, wherein the suspension is suitable for direct infusion into a patient in need of such infusion.

34. The pRBC storage solution according to claim 1, wherein the one or more inorganic phosphates is present at a total concentration of from about 1 mM to about 200 mM.
